(19) United States
(12) Patent Application Publication
(10) Pub. No.: US 2026/0279585 A1
(43) Pub. Date: Sep. 17, 2026

(54) SYSTEM AND METHOD FOR NON-EOSINOPHILIC ASTHMA

(71) Applicant: Norton (Waterford) Limited, Waterford (IE)

(72) Inventors: Katja Sagalovich, Netanya (IL); Yarden Rachamim, Netanya (IL); Michael Reich, Netanya (IL)

(73) Assignee: Norton (Waterford) Limited, Waterford (IE)

(21) Appl. No.: 19/470,753

(22) PCT Filed: Apr. 11, 2024
(86) PCT No.: PCT/EP2024/059803

§ 371 Date: Sep. 29, 2025

Related U.S. Application Data
(60) Provisional application No. 63/458,747, filed on Apr. 12, 2023.

(51) Int. Cl. G16H 50/30; G16H 10/40; G16H 10/60; G16H 50/20; G16H 70/40

(52) U.S. Cl. CPC G16H 50/30; G16H 10/40; G16H 10/60; G16H 50/20; G16H 70/40

(57) ABSTRACT

A system may be configured for determining risk of an asthma exacerbation in a patient with non-eosinophilic asthma (NEA). The system may receive patient data for a patient. The patient data may comprise diagnostic data relating to the patient, laboratory data relating to the patient, pharmaceutical data relating to the patient, and procedure data relating to the patient. The system may also apply a trained model to the patient data to determine risk of an asthma exacerbation of the patient. The system may further generate a notification, via a display device, indicating the risk of the asthma exacerbation of the patient.

### BACKGROUND

Asthma is a chronic disease of the lungs that is characterized by airway inflammation, which causes swelling and excess mucous production, and leads to symptoms of intermittent wheezing, dyspnea, coughing and chest tightness. These symptoms occur in combination with variable expiratory airway obstruction. Clinical diagnosis of asthma is usually established based on the presence of symptoms as well as documented variability in limitations of expiratory airflow. At present, asthma is a major chronic disease affecting approximately 334 million people worldwide. As such, asthma places a heavy burden on societal, financial and health-care levels of nations around the world.

Asthma can be broadly classified as eosinophilic asthma (EA) or noneosinophilic asthma (NEA) on the basis of airway or peripheral blood cellular profiles, with approximately 50% of patients with severe asthma falling into each category. The term EA generally refers to the clinical inflammatory phenotype of asthma wherein a significant number of sputum, airway, and/or blood eosinophils are present in the airways. Sputum cellular profiles are believed to directly reflect lung inflammation and therefore are the preferred method used in asthma research to determine EA. Sputum eosinophils should be reported as a percentage of total cells from either a whole expectorate or sputum plug. Sputum eosinophil levels of greater than 2 to 3% have been used to define EA.

Conversely, NEA represents symptomatic asthma in the absence of eosinophilic airway inflammation, and therefore the lack of eosinophilia, by default, has been used to define NEA. More specifically, NEA is defined by an eosinophil count less than 2% in sputum. While patients with NEA may demonstrate low numbers of eosinophils, the dominant inflammatory cell type are not eosinophils, but instead includes neutrophils, mixed granulocyte inflammatory cells, or very few inflammatory cells, termed paucigranulocytic inflammation. NEA has been associated with environmental and/or host factors, such as smoking cigarettes, pollution, work-related agents, infections, and obesity. These risk factors, alone or in conjunction, can activate specific cellular and molecular pathways leading to non-type 2 inflammation.

Significant progress has been made in the understanding of EA, and the mechanisms driving EA are becoming clearer. As a result, several targeted therapies are available for eosinophilic disease. By contrast, much less attention has been given to the underlying mechanisms that drive NEA, and it remains poorly understood. Many patients with NEA respond poorly to standard asthma treatments, especially to inhaled corticosteroids. This can lead to a higher severity of disease and more difficult-to-control asthma, which can be life-threatening for some patients.

Since most current and forthcoming biologic therapies specifically target type 2 asthma phenotypes, such as uncontrolled severe eosinophilic or allergic asthma, there is a striking lack of effective treatments for uncontrolled non-type 2 asthma. Given the limited understanding of what drives NEA, and the underlying mechanistic complexity of this phenotype, any analyses of NEA are likewise limited. As a result, there is a lack of available therapies for the NEA patient population. There is thus an urgent need to conduct clinical research and uncover knowledge about the mechanisms and other factors underlying NEA, as well as the development of the ability to predict NEA, for which no targeted biologic or other therapies are yet available and outcomes from studies are still lacking.

### SUMMARY

A method for determining risk of an asthma exacerbation, for example, in patients with non-eosinophilic asthma (NEA), may include receiving patient data for a patient. For example, the patient data may comprise diagnostic data relating to the patient, laboratory data relating to the patient, pharmaceutical data relating to the patient, and procedure data relating to the patient. In an example, the method may include applying a trained model to the patient data to determine risk of an asthma exacerbation of a patient (for example, a patient with NEA). The method may further include generating a notification, via a display device, indicating the risk of the asthma exacerbation of the patient (for example, the patient with NEA).

Provided herein are methods and systems for determining the risk of an asthma exacerbation in a patient (e.g., an individual and/or a user) (for example, in a patient with NEA). A method may include receiving patient data for a patient. The patient data may include any combination of diagnostic data relating to the patient, laboratory data relating to the patient, pharmaceutical data relating to the patient, and/or procedure data relating to the patient. The method may include applying a trained model to the patient data to determine risk of an asthma exacerbation of a patient, and generating a notification, via a display device, that indicates the risk of the asthma exacerbation of the patient. The display device may be comprised within a mobile device or a personal computer of the patient or a health care provider of the patient.

In some examples, the notification may indicate that the patient is at risk of the asthma exacerbation within a time period (e.g., within a year or a month). In some examples, the method may include generating a notification that provides a treatment recommendation based on an identified risk group of the patient (e.g., a low-risk group, a medium-risk group, and/or a high-risk group).

In some examples, the method may include pre-processing the diagnostic data to generate, for each of a plurality of diagnostic categories, an indication of whether or not the patient has been diagnosed with the diagnostic category and/or how many times the diagnosis was made. The diagnostic categories may be defined by the International Classification of Diseases 10th Revision (ICD-10) code system.

In some examples, the method may include pre-processing the laboratory data to determine a statistical measure for one or more of a plurality of different laboratory tests for the patient. The statistical measure of each of the plurality of different laboratory tests for the patient may include one or more of a minimum value for the laboratory test, a maximum value for the laboratory test, a mean for the laboratory test, and/or a difference between a first result and a last result for the laboratory test. In some examples, the laboratory data may be defined by a code system, such as Logical Observation Identifiers, Names, and Codes (LOINC). For instance, in some examples, pre-processing the laboratory data may include removing laboratory tests with no data from the laboratory data, splitting the LOINC of each laboratory test into a tuple (e.g., LOINC, UNIT, NAME, VALUE-mean), calculating for each LOINC, a mean of the VALUE-mean and a standard deviation, and excluding each tuple that has a standard deviation count that is greater than a predetermined number.

In some examples, the method may include pre-processing the pharmaceutical data to determine a number of unique medication prescriptions prescribed to the patient. For example, pre-processing the pharmaceutical data may include determining a count of total prescriptions, a count of prescriptions of asthma rescue medication, and a count prescriptions of asthma anti-body medicine. In some examples, pre-processing the pharmaceutical data may include generating, for each of a plurality of medication prescriptions, an indication of whether the patient is prescribed the medication. The pharmaceutical data may be associated with a code based on a National Drug Code (NDC).

In some examples, the method may include pre-processing the procedure data to determine a number of procedures for the patient in an identified subset of procedure group types. The procedure data may be associated with a code based on a Current Procedural Terminology (CPT) code.

In some examples, the method may include pre-processing the patient data as a feature vector that includes a concatenation of the patient data and metadata, where the metadata may include demographic data of the patient. The demographic data may include at least one of age, ethnicity, or gender.

In some examples, the method may include training the model. Training the model may include receiving patient data for each patient of a plurality of patients. The patient data for each patient may include any combination of diagnostic data relating to the respective patient, laboratory data relating to the respective patient, pharmaceutical data relating to the respective patient, and/or procedure data relating to the respective patient. The patient data may include an indication of whether the patient had an asthma exacerbation during a baseline time period (e.g., the previous year). Training the model may include inputting the patient data into a machine learning model to develop the trained model. To develop the predictive model, machine learning techniques may be applied to one or more of patient data. In an example, machine learning techniques may comprise gradient-boosting trees. In some examples, the machine learning model may include an Extreme Gradient Boosting (XGBoost) algorithm.

A method may be used to determine patients that are best suited for participation in a clinical trial. For instance, the methods and system described herein may be used to train and/or use a machine learning model that has been specifically designed to determine the patients that are best suited for participation in a clinical trial (e.g., patients with the highest risk of asthma exacerbation, are least likely to leave the trail early, are most likely to be similarly situated and benefit from the trial, are those who would provide the most accurate results for the trial, and/or the like). For example, a method for determining risk of an asthma exacerbation may include receiving patient data for each patient of a plurality of patients. The patient data for each patient may include any combination of diagnostic data relating to the patient, laboratory data relating to the patient, pharmaceutical data relating to the patient, and/or procedure data relating to the patient. The method may include applying a trained model to the patient data to determine risk of an NEA-asthma exacerbation of a patient (e.g., each patient). The method may include generating a notification that identifies a subset of the plurality of patients for participation in a clinical trial, wherein the identification of the subset is based on an identification of a high-risk group of the patients.

In some examples, the method may include identifying a plurality of risk groups for the plurality of patients. The notification may indicate a risk group out of the plurality of risk groups of the patient. The plurality of risk groups may include any combination of a low-risk group, a medium-risk group, and/or a high-risk group. The low-risk group may be defined as patients having a likelihood of an asthma exacerbation below a lower threshold. The medium risk group may be defined as patients having a likelihood of an asthma exacerbation above the lower threshold and below an upper threshold. The high-risk group may be defined as patients having a likelihood of an asthma exacerbation above the upper threshold. In some examples, the method may include determining, using the trained model, a risk value for the patient, and comparing the risk value to the lower threshold and upper threshold to determine the risk group of the patient.

### BRIEF DESCRIPTION OF THE DRAWINGS

FIG. 1 is a diagram of an example system for collecting data from various sources and generating a predictive model capable of determining the risk of an asthma exacerbation of a patient with NEA.

FIG. 2 is a graph of an exemplary Receiver Operating Characteristic (ROC) curve based on results of the predictive model in FIG. 1.

FIG. 3 is a block diagram that illustrates an example of a computing device of the example system shown in FIG. 1.

FIG. 4 is a block diagram of that illustrates an example of an external device.

FIG. 5 is a flow diagram that illustrates an example process for training an example model to determine risk of an asthma exacerbation in a patient with NEA.

FIG. 6 is a flow diagram that illustrates an example process for using an example of a trained model to generate a notification.

### DETAILED DESCRIPTION

It should be understood that the detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present invention will become better understood from the following description, appended claims, and accompanying drawings. It should be understood that the Figures are merely schematic and are not drawn to scale. It should also be understood that the same reference numerals are used throughout the figures to indicate the same or similar parts.

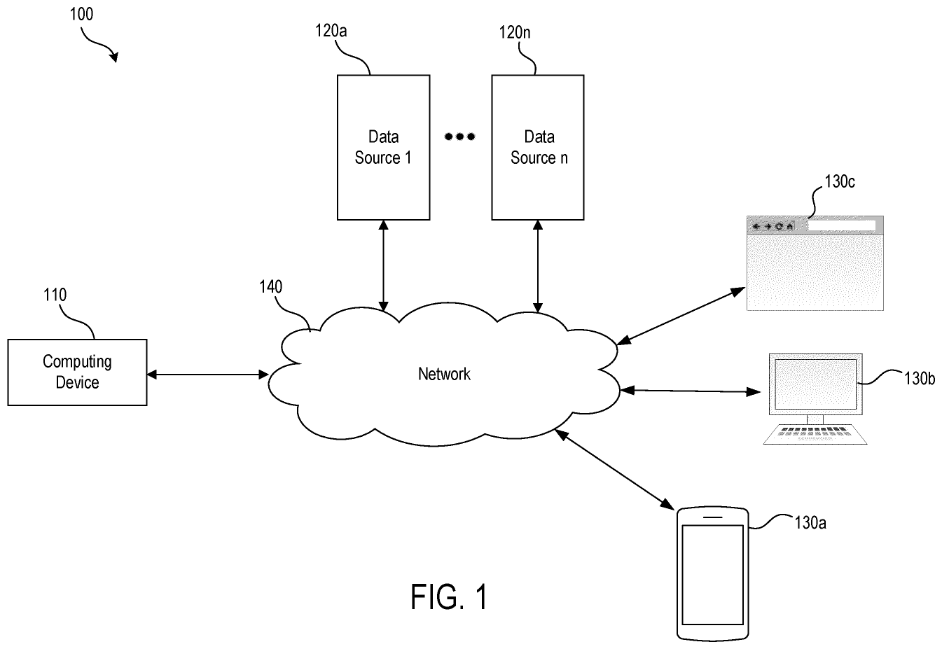

FIG. 1 is a diagram of an example system for collecting data from various data sources and generating a predictive model capable of determining the risk of an asthma exacerbation of a patient (e.g., a user or an individual). The patient may be a patient with NEA (e.g., a patient who has been diagnosed with NEA). The system 100 may include a computing device 110, one or more data sources 120a-120n, and one or more remote client devices 130a-130c.

The computing device 110, one or more data sources 120a-120n, and one or more remote client devices 130a-130c may be connected via a network, such as a public and/or private network 140 (e.g., any combination of the Internet, a cloud network, and/or the like). The network 140 may comprise any suitable network over which computing device 110, one or more data sources 120a-120n, and one or more remote client devices 130a-130c may communicate. The network 140 may include a wired and/or wireless communication network. Example wireless communication networks may be comprised of one or more types of RF communication signals using one or more wireless communication protocols, such as a cellular communication protocol, a WIFI communication protocol, and/or another wireless communication protocol.

The computing device 110 may comprise a server or combination of servers that may be configured to receive data from various sources, such as the data sources 120a-120n. In some examples, the computing device 110 can comprise a mobile device, such as a smartphone, laptop, or tablet. The computing device 110 may comprise an operational system or subsystem and an analytical system or subsystem. The computing device 110 may generate (e.g., train) a predictive model capable of determining the risk of an asthma exacerbation of a patient with NEA based on the data received from the data sources 120a-120n. In an example, the computing device may perform training on the predictive model to allow the model to determine risk of an asthma exacerbation on a patient with NEA, for example, based on the data received from the data sources 120a-120n. Alternatively or additionally, the computing device 110 may use the trained predictive model that is capable of determining the risk of an asthma exacerbation of a patient based on the data received from the data sources 120a-120n. One example of the computing device 110 may be a digital health platform (DHP). Examples of a DHP are described in greater detail in U.S. Patent Publication No. US 2022/0148730 A1, published May 12, 2022, entitled Inhaler System, the entire disclosure of which is hereby incorporated by reference.

Initially, asthma patients may undergo a blood test to characterize their asthma as eosinophilic or noneosinophilic, such as a blood test to measure levels of eosinophils. The results of such a blood test may indicate the blood concentration of eosinophils (i.e., eosinophils per mcL of blood). For the exacerbation prediction method described below, the patient population may be filtered so as to only analyze data for asthma patients (for example, patients with NEA). As such, for the exacerbation prediction method described below, data will only be analyzed for patients with a documented eosinophilic blood concentration less than a predetermined threshold. In one embodiment, patients must have a blood test documenting an eosinophilic blood count of less than 250/mcL of blood for exacerbation prediction according to the below-described methods and systems. Where patients have undergone multiple blood tests, blood test data may show a mean eosinophilic blood count of less than 250/mcL of blood for exacerbation prediction according to the below-described methods and systems.

The computing device 110 may be configured to receive data from the data sources 120a-120n via the network 140. The data sources 120a-120n may store a variety of data, such as patient data for the confirmed asthma patients with NEA. Though the data sources 120a-120n may also store data for asthma patients without NEA, the data for confirmed asthma patients with NEA may be considered by the model as described herein. The patient data may be electronic medical record (EMR) data (e.g., baseline information). Alternatively or additionally, the patient data may be real-world evidence (RWE) data from insurance claims. For example, the patient data may include diagnostic data relating to the patient, laboratory data relating to the patient, pharmaceutical data relating to the patient, and/or procedure data relating to the patient. Accordingly, in some examples, the computing device 110 may access a first data source 120a that comprises diagnostic data relating to the patient, a second data source 120b that comprises laboratory data relating to the patient, a third data source 120c that comprises pharmaceutical data relating to the patient, and/or a fourth data source 120d that comprises procedure data relating to the patient. In some examples, some or all of the diagnostic data, laboratory data, pharmaceutical data, and/or procedure data may be drawn from the same source.

The patient data may span (e.g., have been collected over) a certain period of time, e.g., a baseline time period. In one example, the baseline time period may be a period of one year prior to a qualifying blood test for eosinophilic concentration, as described above. When more than one qualifying blood test for eosinophilic concentration exists for a patient, the baseline time period may be a period of one year prior to the most recent blood test. For example, the patient data may pertain to (e.g., only pertain to) the baseline time period. In some examples, the computing device 110 may apply the model to predict the likelihood of an asthma exacerbation for a patient with NEA, for example, during a follow-up period of a certain length of time, for example, during a one-year follow up period based on the received patient data. In an embodiment, the computing device 110 may characterize one or more predictors of an asthma exacerbation using the predictive model.

As noted above, in some examples, the patient data may include various categories or groups of data, such as diagnostic data relating to the patient, laboratory data relating to the patient, pharmaceutical data relating to the patient, and/or procedure data relating to the patient. It is contemplated that the baseline time period for each category of the patient data can be the same. In an example, each group of patient data may differ from another group, for example, in terms of data types, amount of information per patient, unit measures, and/or the like. In an example, each group of patients may differ within itself, for example, between patients. Further, the amount of data, the unit measures, and/or the type of data within a single group (e.g., laboratory data) may be different for different patients. Therefore, the computing device 110 may be configured to model highly complex and heterogeneous data from various data sources 120a-120n.

The computing device 110 may pre-process the patient data, for example, to homogenize the format or structure of the patient data within each category. The pre-processed patient data may, for example, be referred to as and/or comprise different factors/attributes that are used by and defined by the machine learning model. In some examples, the computing device 110 may pre-process the patient data using a unique code system for each group of data. For instance, a different code system may be provided for each group of data types. In an example, the diagnosic data may use a code system comprising International Classification of Diseases codes (e.g., ICD9, ICD10 codes, and/or the like). The laboratory data may use a code system comprising Logical Observation Identifiers Names and Codes (LOINC). The pharmaceutical data may use a code system comprising National Drug Code (NDC). The procedure data may use a code system comprising Current Procedural Terminology (CPT). Though one combination of unique code systems is described, the present disclosure is not intended to be limited to such. In an example, each data group may require different preprocessing steps.

As noted above, the computing device 110 may pre-process the diagnostic data. For example, the computing device 110 may pre-process the diagnostic data to determine the plurality of diagnostic categories. The diagnostic categories may include any combination of the following: an indication of whether the patient had unspecified asthma (uncomplicated) (e.g., during a baseline time period), how many times a diagnosis was made during the baseline time period, an indication of whether the patient had moderate persistent asthma with (e.g., acute) exacerbation (e.g., during a baseline time period), an indication of whether the patient had severe persistent asthma with status asthmaticus (e.g., during a baseline time period), an indication of whether the patient had mild persistent asthma (e.g., during a baseline time period), an indication of whether the patient had vasomotor and allergic rhinitis (e.g., during a baseline time period), an indication of whether the patient had a period of gestation (e.g., during a baseline time period), an indication of whether the patient had an asthma exacerbation during a baseline time period (e.g., during the previous year), etc. The indication may be binary (e.g., 1 for yes and 0 for no). As noted above, the baseline period may be one year, such as the year preceding a qualifying eosinophilic blood test.

The diagnostic data may be coded using the ICD-10 code system. In an example, the ICD-10 code system may display any combination of the following: category, etiology, location, laterality, and/or extension. The computing device 110 may pre-process the diagnostic data to generate, for each of a plurality of diagnostic categories, an indication of whether or not the patient has been diagnosed with the diagnostic category, and how many times a particular diagnosis was made.

As noted above, the computing device 110 may pre-process the laboratory data. The laboratory data for a patient may include any combination of the following: average potassium level (e.g., yearly), average calcium level, average glucose level, minimum glucose level, maximum platelets level, and/or the like obtained during a baseline time period. The baseline period may be one year, such as the previous year.

The computing device 110 may pre-process the laboratory data, for instance, using a statistical approach. For example, the computing device 110 may pre-process the laboratory data and identify a subset (e.g., only a subset) of test results, such as a subset of 100 test results. In an example, lab results with no data or 0 as the result (e.g., except the tests for which 0 may be a possible result) may be removed. For example, a blood hemoglobin lab result having either no data or with 0 as the result may be removed. In a further example of laboratory data pre-processing, in an example the mean of the value-mean and standard-deviation may be calculated for the laboratory data. In an example, outliers may be removed. For example, each tuple with a standard deviation count greater than 3 may be excluded. In an example, calculations may be performed for each patient and laboratory data result, comprising, for example, minimum, maximum, mean, and the difference between the first and last result.

The laboratory data may be coded using the LOINC system. LOINC may be a database and universal standard for identifying medical laboratory observations.

The computing device 110 may pre-process pharmacy data. In an example, the computing device 110 may pre-process the pharmacy data to define any combination of the following features: count of total prescriptions; count of unique medication prescriptions (e.g., labeler and product code); an indication for each one of a plurality of medication prescriptions (e.g., 10 medication prescriptions); a count prescription of asthma rescue medication (e.g., beta2 agonist); and/or a count prescriptions of asthma anti-body medicine. In some examples, the pharmacy data be coded a National Drug Code (NDC), such as, for example, a unique 11-digit, 3-segment number, and a universal product identifier for human drugs. In some examples, the pharmacy data may be based upon a baseline time period. The baseline period may be one year, such as the year, such as the year preceding a qualifying eosinophilic blood test.

The computing device 110 may pre-process the procedure data. The procedure data for a patient may include a combination of the following: an indication of whether the patient had one or more surgeries, a count of pulmonary procedures received (e.g., which may include pulmonary diagnostic testing, therapies, and/or ventilator management), a number of office visits, a number of and/or type of blood tests, an indication of whether the patient has had physical therapy, an indication and/or number of instances where the patient was admitted to the emergency department (e.g., level 4), and/or the like. In some examples, the procedure data may be based upon a baseline time period. The baseline period may be one year, such as the previous year, such as the year preceding a qualifying eosinophilic blood test.

The procedure data may be coded a current procedural terminology (CPT). For example, CPT may describe how to report procedures and/or services. For example, CPT may have 5 digits. In an example, each CPT code may be in a specific range. Each range may represent a specific group type. In an example, the computing device 110 may pre-process the procedure data and only implement a subset of the group types within a prediction model. In an example, the model may count group procedures experienced by each patient.

The computing device 110 may train the machine learning model using the pre-processed data and/or the processed data. In some examples, the computing device 110 may generate an identifier for each patient that indicates whether the patient with a qualifying eosinophilic blood test as described above had an asthma exacerbation (e.g., during the baseline period). The computing device 110 may use this identifier when training the model. For example, the computing device 110 may generate a feature vector for each patient within the training data set, where the feature vector may be a concatenation of one or more electronic medical record groups and additional meta-data (e.g., age, ethnicity, and/or the like). In an example, the computing device 110 may train the machine learning model using the patient data from a plurality of patients, the identifier that indicates whether the patient had an asthma exacerbation, and/or other patient information for each patient (e.g., age, ethnicity, etc.). In some instances, the model may be an algorithm based on ensemble of decision trees and/or used for regression, classification, and/or ranking problems. In an example, data modeling may be performed wherein the model used may be Extreme Gradient Boosting (XG-Boost). Although, as described in more detail below, other models may be implemented.

As an alternative to and/or in addition to training the model, the computing device may apply the trained model to patient data (e.g., for a new patient, i.e., a patient whose patient data was not used to train the model) to determine risk of an asthma exacerbation of the patient. The new patient may have NEA. As stated above, patient data may be initially filtered such that the trained model will only be applied to data of patients having a qualifying eosinophilic blood test, such as below 250 eosinophils/mcL of blood. The model may output an indication that indicates a risk of the asthma exacerbation of the patient (e.g., the new patient), for example, over a future time period (e.g., within the next month(s), year, etc.). In some examples, the indication may be binary (e.g., 1 if the patient is at risk of an asthma exacerbation, and 0 if the patient is not at risk of an asthma exacerbation). In other examples, the indication may indicate whether the patient is within one of a plurality of risk groups. The plurality of risk groups may include any combination of a low-risk group, a medium-risk group, and/or a high-risk group. The low-risk group may be defined as patients having a likelihood of an asthma exacerbation below a lower threshold. The medium risk group may be defined as patients (e.g., patients with NEA) having a likelihood of an asthma exacerbation above the lower threshold and below an upper threshold. The high-risk group may be defined as patients (e.g., patients with NEA) having a likelihood of an asthma exacerbation above the upper threshold. In some examples, the computing device 110 may determine, using the trained model, a risk value for the patient. The risk value may be compared to the lower threshold and upper threshold to determine the risk group of the patient.

The computing device 110 may generate a notification indicating the risk of the asthma exacerbation of the patient (e.g., a binary indication, an indication of a risk group, risk value, etc.). For example, as noted above, the system 100 may include one or more remote client devices 130a-130c. The client devices 130a-130c may comprise, inter alia, a display device. In some examples, the computing device 110 may be configured to generate the notification via the display device of a client device 130a-130c. The notification may indicate the percentage likelihood of exacerbations, the risk group for exacerbation that the patient belongs to, risk assessment information (e.g., a risk group and/or clinical trial notification type), and/or the like. The client device 130a may comprise a web-portal that, for example, may be presented via a server or other computer. The client device 130b may be a personal computer that, for example, may be associated with a physician or other health care provider, a patient or person related to a patient, pharmaceutical company, etc. The client device 130c may be a mobile device, such as a smartphone, laptop, or tablet, that may be associated with a physician or other health care provider, a patient or person related to a patient, pharmaceutical company, etc.

The predictive performance of the algorithms may be evaluated using receiver operating characteristics (ROC) curve of sensitivity versus specificity. In an example, the ROC area under the curve (AUC) value may represent the capability of the model to separate between classes. In an example, a values for AUC may fall between 0 and 1, with 1 representing perfect performance of the model. In an example, the performance of a model, which may be determined by an ROC curve, may range in value between 0.5 and 1. For example, predicting exacerbation in the follow up year may yield an AUC value of 0.72. In an example, predicting exacerbation at the first month of the follow up year may yield an AUC value of 0.75.

The computing device 110 may train a machine learning model and/or apply a trained machine learning model that includes any combination of the following features (e.g., for each patient); gender; age; one or more lab results (e.g., blood count, ALT, glucose, creatinine, and/or the like); one or more prescriptions (e.g., asthma controllers, opioid analgesic and cough suppressants, PPI inhibitors for acid reflux and ulcers, high cholesterol treatment, and/or the like); one or more visit diagnosis (e.g., asthma-related, primary hypertension, diabetes type 2, cholesterol-related disorders, and/or the like); and/or one or more procedures (e.g., a number of office visits, a number of and/or type of blood tests, an indication of whether the patient has had physical therapy, an indication and/or number of instances where the patient was admitted to the emergency department (e.g., level 4), and/or the like). In an example, the computing device 110 may generate a score (e.g., a ratio) based on the weight of a feature (e.g., the impact of the that the feature has) in the operation of the model.

Table 1 is one, non-limiting example, of the particular factors/attributes, (e.g., the patient data) that may be applied by the computing device 110 (e.g., using the machine learning model) when determining a risk of an asthma exacerbation of a patient (for example, a patient with NEA). It should be noted that weights associated with each of the listed factors/attributes have been omitted. As noted herein, the weighting may be a byproduct of the machine learning algorithm, and for example, may indicate the relative significant of the particular factor/attribute (e.g., the patient data) when determining the risk of an asthma exacerbation of a patient (e.g., a patient with NEA).

TABLE 1
Example Factors/Attributes when determining an Individualized Score.
Factor/Attribute

| | |
|---|---|
| Diagnosis | Unspecified asthma, uncomplicated |
| | Moderate persistent asthma with (acute) exacerbation |
| | Severe persistent asthma with status asthmaticus |
| | Mild persistent asthma |
| | Vasomotor and allergic rhinitis |
| | Weeks of gestation |
| | Encounter for supervision of normal pregnancy |
| | Essential (primary) hypertension |
| Procedures | Pulmonary (Pulmonary Diagnostic Testing and Therapies, |
| | ventilator management) |
| | Surgery |
| | Nursing facility |
| Prescriptions | Unique medicine prescriptions count |
| | All prescriptions count |
| | Systolic blood pressure |
| Lab results | Average yearly potassium level |
| | Average calcium level |
| | Average glucose level |
| | Minimum glucose level |
| | Maximum platelets level |

For instance, in some specific example, the computing device 110 may train a machine learning model and/or apply a trained machine learning model that includes any combination of the following features (e.g., for each patient) (e.g., based on the patient data collected from the data sources 120a-120n): a diagnosis of unspecified asthma (e.g., uncomplicated); a unique medicine prescriptions count; a diagnosis of moderate persistent asthma with acute exacerbation; a count of all prescriptions taken; a count of pulmonary procedures received (e.g., which may include pulmonary diagnostic testing, therapies, and/or ventilator management); a diagnosis of severe persistent asthma with status asthmaticus; a diagnosis of mild persistent asthma; a diagnosis of vasomotor and allergic rhinitis; an asthma exacerbation indication last year; an asthma rescue medicine prescriptions count; a diagnosis of weeks of gestation; the a surgical procedure; average potassium level (e.g., yearly); a diagnosis of encounter for supervision of normal pregnancy; a procedure performed in a nursing facility; a diagnosis of essential (e.g., primary) hypertension; lab results of average calcium level; lab results of average glucose level, lab results of minimum glucose level; lab results of maximum platelets level; and/or the like, and/or a correlation between any of the factors provided above.

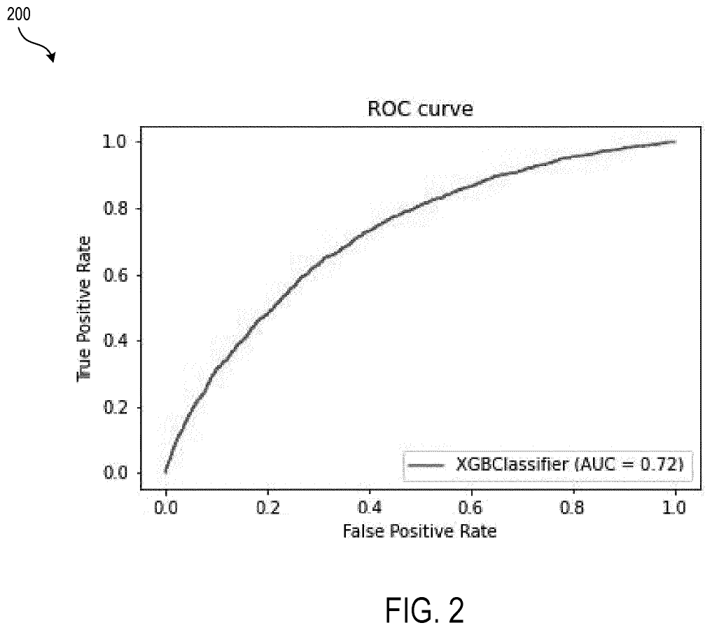

FIG. 2 shows an example Receiver Operating Characteristic (ROC) curve 200 that, for example, may be based on and/or evaluate the diagnostic performance of a test, and/or evaluate the accuracy of a test. The ROC curve analysis of the model assesses the quality of the model by plotting the true positive rate against the false positive rate. In an example, the ROC curve 200 may be based on and/or produced using results of an asthma prediction model, such as the model trained and operated by the computing device 100 in FIG. 1. In an example, the ROC curve 200 and/or the Area Under the Curve (AUC) may represent results of an example XGBoost (XGB) classifier. It may be appreciated that ROC curve 200 of true positive rate and false positive rates shown in FIG. 2 are merely examples, and may vary based on the computing device 100, the training and operation of the NEA prediction model, the data and the data sources 120a-120n, and/or the like. Referring back to FIG. 1, and as noted above, the computing device 100 may train and/or use a model. The model may be a linear model or a non-linear model. The model may be a machine learning model. A supervised model, such as a supervised machine learning model, may be used. In an example, the model is constructed using an XG-Boost algorithm. Other suitable techniques, such as building a neural network or a deep learning model may also be contemplated by the skilled person.

The computing device 100 may train and/or use a predictive model, where the predictive model may use any combinations of algorithms, including logistic regression, random forest, and gradient-boosting trees. The predictive model may be developed to determine the probability of the asthma exacerbation. The supervised machine learning technique, Gradient Boosting Trees, may be used to solve a classification problem (whether or not there is an exacerbation in the upcoming x days (exacerbation period)). The Gradient Boosting Trees technique may build a single strong learner model in an iterative fashion by using an optimization algorithm to minimize some suitable loss function (e.g., a function of the difference between estimated and true values for an instance of data). The optimization algorithm may use a training set of known values of a variable (whether or not there will be an exacerbation in the upcoming x days) and their corresponding values of predictors (e.g., a list of the features) to minimize the expected value of the loss function. The learning procedure may consecutively fit new models to provide a more accurate estimate of the response variable.

To develop the predictive model, machine learning techniques may be applied to a combination of patient data prior to (and including) the day of the prediction, including patient data from a baseline time period. A feature engineering process may be conducted to determine the most relevant features of the model. For example, the machine learning model may comprise gradient boosted decision trees and/or an XG-Boost implementation of gradient-boosting. In an example, gradient boosted decision trees may combine weak learners to minimize the loss function. For example, regression trees may be used to produce real values for splits and/or to be added together. The weak learners may be constrained, for example, to a maximum number of layers, a maximum number of nodes, a maximum number of splits, etc. Trees may be added one at a time to the machine learning algorithm and/or existing trees may remain unchanged. A gradient descent procedure may be used to minimize loss when adding trees. For example, additional trees may be added to reduce the loss (e.g., follow the gradient). In an example, the additional tree(s) may be given parameters and those parameters may be modified to reduce the loss. In an example, the XGBoost algorithm may comprise an implementation of gradient boosted decision trees that may be designed for speed and/or performance.

The XGBoost algorithm may (e.g., automatically) handle missing data vales, support parallelization of tree construction, and/or continued training. The Gradient Boosting Trees technique may produce a prediction model in the form of an ensemble (e.g., multiple learning algorithms) of base prediction models, which are decision trees (e.g., a tree-like model of decisions and their possible consequences). The XGBoost algorithm may build a single strong learner model in an iterative fashion by using an optimization algorithm to minimize some suitable loss function (e.g., a function of the difference between estimated and true values for an instance of data). The optimization algorithm may use a training set of known values of the response variable (e.g., yes/no exacerbation in the upcoming x days) and their corresponding values of predictors (e.g., the list of the features and engineered features) to minimize the expected value of the loss function. The learning procedure may consecutively fit new models to provide a more accurate estimate of the response variable.

Although described primarily in the context of an XGBoost algorithm, the computing device 110 may use other machine learning models, such as an unsupervised learning method (e.g., a clustering method, such as a k-means or c-means clustering method) and/or a supervised learning method (e.g., gradient boosted decision trees). As an example, the computing device 110 may use a gradient descent or a stochastic gradient descent learning method. A supervised learning method may use labeled training data to train the machine learning algorithm. As training data is received, the supervised learning method may adjust weights until the machine learning algorithm is appropriately weighted. The supervised learning method may measure the accuracy of the machine learning algorithm using a loss function. The supervised learning method may continue adjusting the weights until the error is reduced below a predetermined threshold.

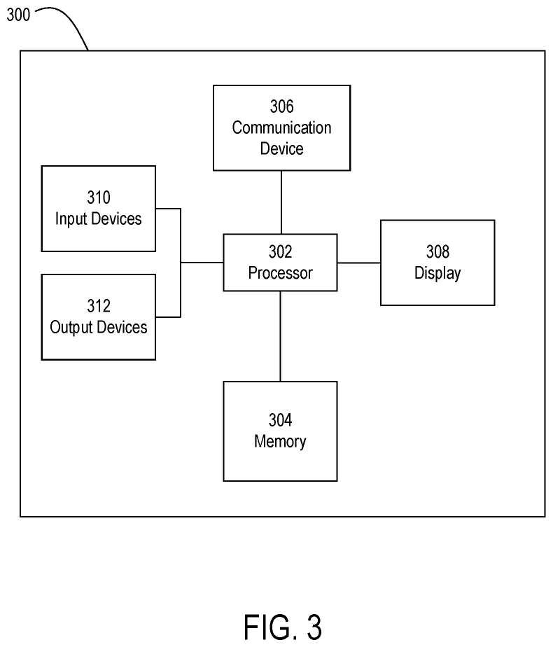

FIG. 3 is a simplified block diagram of an example device 300. The device 300 may be an example of a computing device (e.g., one or more servers), such as the computing device 110 of the system 100 of FIG. 1. Alternatively or additionally, the device 300 may be an example of a client device, such as the client devices 130a-130c of the system 100 of FIG. 1. In such instances, the device 300 may include a personal computer, such as a laptop or desktop computer, a tablet device, a cellular phone or smartphone, a server, or another type of client device. The device 300 may be configured to develop, train, or use one or more machine learning models described herein. As shown by FIG. 3, the device 300 may comprise a processor 302, a memory 304, a communication device 306, a display 308, one or more input devices 310, and/or one or more output devices 312. It should be appreciated that the device 300 may include fewer or more components than those shown in FIG. 3.

The processor 302 may include one or more general purpose processors, special purpose processors, conventional processors, digital signal processors (DSPs), microprocessors, integrated circuits, a programmable logic device (PLD), application specific integrated circuits (ASICs), or the like. The processor 302 may perform signal coding, data processing, image processing, power control, input/output processing, and/or any other functionality that enables the device 300 to perform as described herein.

The processor 302 may store information in and/or retrieve information from the memory 304. The memory 304 may include a non-removable memory and/or a removable memory. The non-removable memory may include random-access memory (RAM), read-only memory (ROM), a hard disk, or any other type of non-removable memory storage. The removable memory may include a subscriber identity module (SIM) card, a memory stick, a memory card, or any other type of removable memory. The memory may be local memory or remote memory external to the device 300. The memory 304 may store instructions which are executable by the processor 302. Different information may be stored in different locations in the memory 304.

The memory 304 may comprise a computer-readable storage media or machine-readable storage media that maintains computer-executable instructions for performing one or more as described herein. For example, the memory 304 may comprise computer-executable instructions or machine-readable instructions that include one or more portions of the procedures described herein. The processor 302 of the device 300 may access the instructions from memory for being executed to cause the processor 302 of the device 300 to operate as described herein. The memory 304 may comprise computer-executable instructions for executing configuration software. For example, the computer-executable instructions may be executed to perform, in part and/or in their entirety, one or more procedures as described herein. Further, the memory 304 may have stored thereon one or more settings and/or control parameters associated with the device 300.

The processor 302 that may communicate with other devices via the communication device 306. The communication device 306 may transmit and/or receive information over a network (e.g., the network 140), which may include one or more other devices. The communication device 306 may perform wireless and/or wired communications. The communication device 306 may include a receiver, transmitter, transceiver, or other device capable of performing wireless communications via an antenna. The communication device 306 may be capable of communicating via one or more protocols, such as a cellular communication protocol, a Wi-Fi communication protocol, Bluetooth®, a near field communication (NFC) protocol, an internet protocol, another proprietary protocol, or any other radio frequency (RF) or communications protocol. The device 300 may include one or more communication devices 306.

The processor 302 may be in communication with a display 308 for providing information to a user. The information may be provided via a user interface on the display 308. The information may be provided as an image generated on the display 308. The display 308 and the processor 302 may be in two-way communication, as the display 308 may include a touch-screen device capable of receiving information from a user and providing such information to the processor 302. The processor 302 may be configured to generate, on the display 308, an indication one or more notifications described herein, such as an indication of the risk of the NEA exacerbation of the patient, the risk category of the patient, etc.

The processor 302 may be in communication with input devices 310 and/or output devices 312. The input devices 310 may include a camera, a microphone, a keyboard or other buttons or keys, a mouse, and/or other types of input devices for sending information to the processor 302. The display 308 may be a type of input device, as the display 308 may include touch-screen sensor capable of sending information to the processor 302. The output devices 312 may include speakers, indicator lights, or other output devices capable of receiving signals from the processor 302 and providing output from the device 300. The display 308 may be a type of output device, as the display 308 may provide images or other visual display of information received from the processor 302.

Although not illustrated, the device 300 may include a power supply. In some examples, the power supply may include one or more batteries. In some examples, the power supply may include an AC to DC power converter. The power supply may be configured to power the processor 302 and the other low voltage circuitry of the device 300.

Although not illustrated, the device 300 may include a GPS circuit (e.g., in instances where the device 300 is a client device). The processor 302 may be in communication with the GPS circuit for receiving geospatial information. The processor 302 may be capable of determining the GPS coordinates of the device 300 based on the geospatial information received from the GPS circuit. The geospatial information may be communicated to one or more other communication devices to identify the location of the device 300.

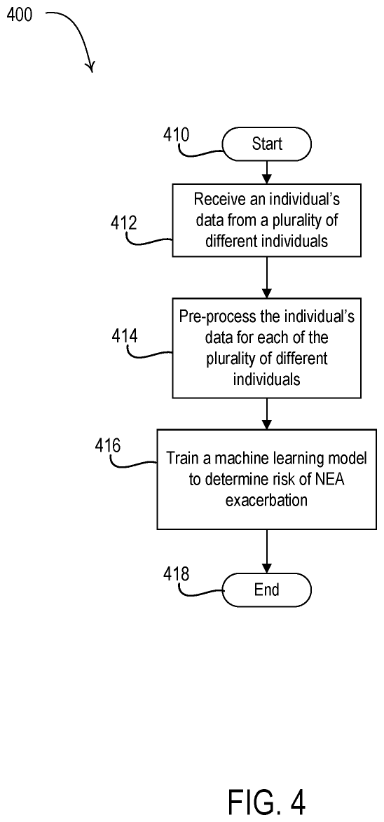

FIG. 4 is a flowchart of a training procedure 400 performed by a device, such as the computing device 110 of the system 100 of FIG. 1 and/or the device 300 (e.g., as a computing device) of FIG. 3. A processor of the computing device may perform the training procedure 400 to train a machine learning model to predict an NEA exacerbation of a patient. The processor may be configured to perform the procedure 400 periodically, for example, in response to an alarm or a schedule that may be configurable. Additionally or alternatively, the processor may be configured to perform the procedure 400 on command (e.g., in response to a command or prompt to perform the procedure 400, such as after new data is received). The procedure 400 may start at 410.

At 412, the processor may receive patient data for a plurality of (e.g., different) patients. For example, the processor may receive the patient data from one or more data sources (e.g., such as the data sources 120a-120n). The patient data may include diagnostic data relating to the patient, laboratory data relating to the patient, pharmaceutical data relating to the patient, and/or procedure data relating to the patient (e.g., as described with reference to FIG. 1). The data may be associated with a plurality of different patients. The processor may filter the data so as to only include in the training data patients with a qualifying eosinophilic blood test (e.g., test results with eosinophilic levels below a predetermined threshold). In one embodiment, only patients with a blood test indicating less than 250 eosinophils/mcL of blood are considered. Alternatively, such filtering of the data may comprise a separate step prior to 412, where the data is filtered prior to being received by the processor. The patient data may span (e.g., have been collected over) a certain period of time, for example, a period of one year prior to the qualifying eosinophilic blood test.

The format of the patient data (e.g., amount of data, the unit measures, and/or the type of data within a single group) may be different for different patients. Further, as noted above, the patient data may be formatted according to one or more different codes (e.g., including different coding formats within a single patient data category, such as diagnostic data). The patient data received from the one or more data sources may be referred to as raw patient data (e.g., patient data that has yet to be preprocessed by the processor for input into the machine learning model).

At 414, the processor may pre-process the patent data for each of the plurality of different patients. For example, the processor may pre-process the patient data (e.g., the raw patient data), for example, to homogenize the format or structure of the patient data within each category. The processor may pre-process the diagnostic data. For example, the processor may pre-process the diagnostic data to determine the plurality of diagnostic categories. The diagnostic categories may include any combination of the following: an indication of whether the patient had unspecified asthma (uncomplicated) (e.g., during a baseline time period), number of asthma diagnoses, an indication of whether the patient had moderate persistent asthma with (e.g., acute) exacerbation (e.g., during a baseline time period), an indication of whether the patient had severe persistent asthma with status asthmaticus (e.g., during a baseline time period), an indication of whether the patient had mild persistent asthma (e.g., during a baseline time period), an indication of whether the patient had vasomotor and allergic rhinitis (e.g., during a baseline time period), an indication of whether the patient had a period of gestation (e.g., during a baseline time period), an indication of whether the patient had an asthma exacerbation during a baseline time period (e.g., during the previous year), etc. The indication may be binary (e.g., 1 for yes and 0 for no). In some examples, the baseline period may be one year, such as the year preceding a qualifying eosinophilic blood test.

The processor may pre-process the laboratory data. The laboratory data for a patient may include any combination of the following: average potassium level (e.g., yearly), average calcium level, average glucose level, minimum glucose level, maximum platelets level, and/or the like. The laboratory data may comprise data for a baseline period, for example one year, such as the previous year. In some examples, the processor may pre-process the laboratory data, for instance, using a statistical approach. In an example, one or more common tests may be applied. For example, the computing device 110 may pre-process the laboratory data and identify a subset (e.g., only a subset) of test results, such as a subset of 100 test results. In an example, lab results with no data or 0 as the result (e.g., except the tests for which 0 may be a possible result) may be removed. For example, a blood hemoglobin lab result having either no data or with 0 as the result may be removed.

The processor may pre-process pharmacy data. In an example, the processor may pre-process the pharmacy data to define any combination of the following features: count of total prescriptions; count of unique medication prescriptions (e.g., labeler and product code); an indication for each one of a plurality of medication prescriptions (e.g., 10 medication prescriptions); a count prescription of asthma rescue medication (e.g., beta2 agonist); and/or a count prescriptions of asthma anti-body medicine. The pharmacy data may comprise data for a baseline period, for example one year, such as the previous year. In some examples, the pharmacy data be coded a National Drug Code (NDC), such as, for example, a unique 11-digit, 3-segment number, and a universal product identifier for human drugs.

The processor may pre-process the procedure data. The procedure data for a patient may include a combination of the following: an indication of whether the patient had one or more surgeries, a count of pulmonary procedures received (e.g., which may include pulmonary diagnostic testing, therapies, and/or ventilator management), a number of office visits, a number of and/or type of blood tests, an indication of whether the patient has had physical therapy, an indication and/or number of instances where the patient was admitted to the emergency department (e.g., level 4), and/or the like. The procedure data may comprise data for a baseline period, for example one year, such as the previous year.

At 416, the processor may train a machine learning algorithm, for example, using training data that includes, inter alia, the pre-processed patient data. The processor may train the machine learning model using any combination of methods described herein, such as those described with reference to FIG. 1. The training data may include any combination of the following: a plurality of events (e.g., a diagnosis of unspecified asthma (e.g., uncomplicated); a unique medicine prescriptions count; a diagnosis of moderate persistent asthma with acute exacerbation; a count of all prescriptions taken; a count of pulmonary procedures received (e.g., which may include pulmonary diagnostic testing, therapies, and/or ventilator management); a diagnosis of severe persistent asthma with status asthmaticus; a diagnosis of mild persistent asthma; a diagnosis of vasomotor and allergic rhinitis; an asthma exacerbation indication last year; an asthma rescue medicine prescriptions count; a diagnosis of weeks of gestation; the a surgical procedure; average potassium level (e.g., yearly); a diagnosis of encounter for supervision of normal pregnancy; a procedure performed in a nursing facility; a diagnosis of essential (e.g., primary) hypertension; lab results of average calcium level; lab results of average glucose level, lab results of minimum glucose level; lab results of maximum platelets level; and/or any of the attributes/factors described herein). The training data may relate to a plurality of different patients.

Additionally or alternatively, the training data may include data received via a self-assessment (e.g., a daily self-assessment provided by the patient, for example, using a client device), one or more third party applications running on the patient device (e.g., Apple Health® app), another patient device (e.g., FitBit®, Apple Watch®, etc.), and/or data received from one or more medical devices, such as an inhaler (e.g., the inhalation parameters (e.g., peak inspiratory flowrate, inhaled volume, inhalation duration, etc.) associated with usage of the inhaler, a number of rescue usage events in a predetermined number of days, a number of missed maintenance usage events in the predetermined number of days, a frequency of rescue usage events, a percent change in an inhalation parameter, or a percent change in inhalation volume. The machine learning algorithm may be trained, at 416, continuously (e.g., hourly, daily, weekly, etc.). For example, additional data received (e.g., after initial training) may be used to train the machine learning algorithm.

At 416, in an example, the processor may train a machine learning model to determine an indication of the risk of the asthma exacerbation of a patient (e.g., a patient with NEA). In some examples, the indication may be a percentage likelihood of NEA exacerbation exacerbations (e.g., over an upcoming time period, such as over the next month(s) or year). In some examples, the the indication may be an identification of a risk group for the patient out of a plurality of risk groups. In some examples, the processor may train the machine learning model to identify a plurality of patients that are best suited for participation in a clinical trial. After training the machine learning model, the training procedure 400 may exit at 418.

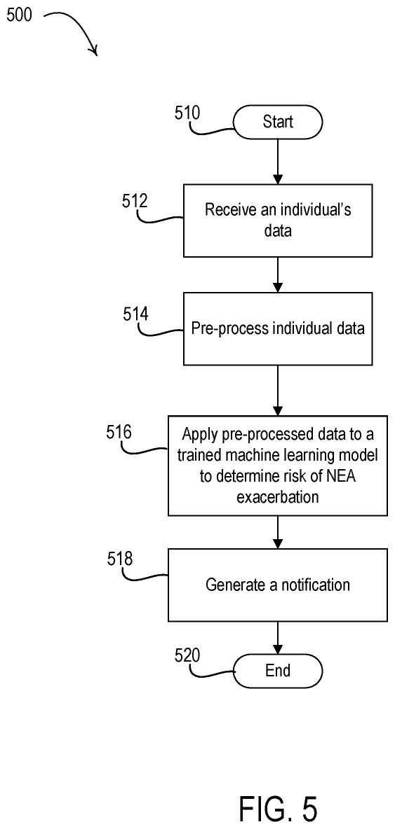

FIG. 5 illustrates an example procedure 500 performed by a computing device, such as the computing device 110 of the system 100 of FIG. 1 and/or the device 300 (e.g., as a computing device) of FIG. 3. A processor of the computing device may perform the procedure 500 to use a machine learning model to generate an indication (e.g., notification) of an asthma exacerbation risk of a patient (e.g., a patient with NEA). The processor may be configured to perform the procedure 500 periodically, for example, in response to an alarm or a schedule that may be configurable. Additionally or alternatively, the processor may be configured to perform the procedure 500 on command (e.g., in response to receiving a command or prompt to perform the procedure 500) and/or in response to receiving new patient data. The procedure 500 may begin at 510.

At 512, the processor may receive patient data for a patient (e.g., a new patient, i.e., a patient whose patient data was not used to train the model). For example, the processor may receive the patient data from one or more data sources (e.g., such as the data sources 120a-120n). The patient data may include diagnostic data relating to the patient, laboratory data relating to the patient, pharmaceutical data relating to the patient, and/or procedure data relating to the patient (e.g., as described with reference to FIG. 1). The processor may filter the data so as to only include in the data (e.g., the training data) patients with a qualifying eosinophilic blood test (i.e., test results with eosinophilic levels below a predetermined threshold). In one embodiment, only patients with a blood test indicating less than 250 eosinophils/mcL of blood are considered. Alternatively, such filtering of the data may comprise a separate step prior to 412, where the data is filtered prior to being received by the processor. The patient data may span (e.g., have been collected over) a certain period of time, for example, a period of one year prior to the qualifying eosinophilic blood test.

The format of the patient data (e.g., amount of data, the unit measures, and/or the type of data within a single group) may be different as compared to the other patient data for this patient (e.g., the new patient) and/or different from the patient data that was used to train the machine learning model. Further, as noted above, the patient data may be formatted according to one or more different codes (e.g., including different coding formats within a single patient data category, such as diagnostic data). The patient data received from the one or more data sources may be referred to as raw patient data (e.g., patient data that has yet to be preprocessed by the processor for input into the machine learning model).

At 514, the processor may pre-process the patent data. For instance, the processor may pre-process the patient data (e.g., the raw patient data), for example, to homogenize the format or structure of the patient data within each category and/or to homogenize the format or structure of the patient data with the patient data that was used to train the machine learning model. The processor may pre-process the diagnostic data. For example, the processor may pre-process the diagnostic data to determine the plurality of diagnostic categories. The diagnostic categories may include any combination of the following: an indication of whether the patient had unspecified asthma (uncomplicated) (e.g., during a baseline time period), the number of asthma diagnoses during the baseline time period, an indication of whether the patient had moderate persistent asthma with (e.g., acute) exacerbation (e.g., during a baseline time period), an indication of whether the patient had severe persistent asthma with status asthmaticus (e.g., during a baseline time period), an indication of whether the patient had mild persistent asthma (e.g., during a baseline time period), an indication of whether the patient had vasomotor and allergic rhinitis (e.g., during a baseline time period), an indication of whether the patient had a period of gestation (e.g., during a baseline time period), an indication of whether the patient had an asthma exacerbation during a baseline time period (e.g., during the previous year), etc. The indication may be binary (e.g., 1 for yes and 0 for no). In some examples, the baseline period may be one year, such as the year preceding a qualifying eosinophilic blood test.

The processor may pre-process the laboratory data. The laboratory data for a patient may include any combination of the following: average potassium level (e.g., yearly), average calcium level, average glucose level, minimum glucose level, maximum platelets level, and/or the like. In some examples, the processor may pre-process the laboratory data, for instance, using a statistical approach. In an example, one or more common tests may be applied. For example, the computing device 110 may pre-process the laboratory data and identify a subset (e.g., only a subset) of test results, such as a subset of 100 test results. In an example, lab results with no data or 0 as the result (e.g., except the tests for which 0 may be a possible result) may be removed. For example, a blood hemoglobin lab result having either no data or with 0 as the result may be removed.

The processor may pre-process pharmacy data. In an example, the processor may pre-process the pharmacy data to define any combination of the following features: count of total prescriptions; count of unique medication prescriptions (e.g., labeler and product code); an indication for each one of a plurality of medication prescriptions (e.g., 10 medication prescriptions); a count prescription of asthma rescue medication (e.g., beta2 agonist); and/or a count prescriptions of asthma anti-body medicine. In some examples, the pharmacy data be coded a National Drug Code (NDC), such as, for example, a unique 11-digit, 3-segment number, and a universal product identifier for human drugs.

The processor may pre-process the procedure data. The procedure data for a patient may include a combination of the following: an indication of whether the patient had one or more surgeries, a count of pulmonary procedures received (e.g., which may include pulmonary diagnostic testing, therapies, and/or ventilator management), a number of office visits, a number of and/or type of blood tests, an indication of whether the patient has had physical therapy, an indication and/or number of instances where the patient was admitted to the emergency department (e.g., level 4), and/or the like.

At 516, the processor may apply the pre-processed data of the patient (e.g., the new patient) to a trained machine learning model to determine a risk of an asthma exacerbation for the patient (e.g., the new patient, who as noted above, may have NEA). The machine learning model may have been trained prior to 516 (e.g., in accordance with the procedure 400 of FIG. 4). The model may generate an indication that indicates a risk of the asthma exacerbation of the patient (e.g., the new patient), for example, over a future time period (e.g., within the next month(s), year, etc.). In some examples, the indication may be binary (e.g., 1 if the patient is at risk of an NEA exacerbation, and 0 if the patient is not at risk of an asthma exacerbation). In other examples, the indication may indication whether the patient is within one of a a plurality of risk groups. The plurality of risk groups may include any combination of a low-risk group, a medium-risk group, and/or a high-risk group. The low-risk group may be defined as patients having a likelihood of an asthma exacerbation below a lower threshold. The medium risk group may be defined as patients having a likelihood of an asthma exacerbation above the lower threshold and below an upper threshold. The high-risk group may be defined as patients having a likelihood of an asthma exacerbation above the upper threshold. In some examples, the processor may determine, using the trained model, a risk value for the patient. The processor may also compare the risk value to the lower threshold and upper threshold to determine the risk group of the patient.

At 518, the processor may generate a notification, for example, indicating the risk of an asthma exacerbation of the patient (e.g., the new patient). The notification may indicate the percentage likelihood of exacerbations, the risk group for exacerbation that the patient belongs to, risk assessment information (e.g., a risk group and/or clinical trial notification type), and/or the like. The notification may be provided via a graphical patient interface (GUI) that is displayed on a display device (e.g., a display device of a client device, such as a client devices 130a, 130b, and/or 130c shown in FIG. 1). In some examples, the notification may be displayed for a practitioner and/or health care professional, for example, to allow them to analyze the patient's data, alert the patient, and/or adjust the treatment regimen of the patient (e.g., increase the patient's dosage, switch the patient's dosage, etc.). In other examples, the notification may be displayed for the patient so they may receive their indication of their risk of an asthma exacerbation. The procedure 500 may exit at 520.

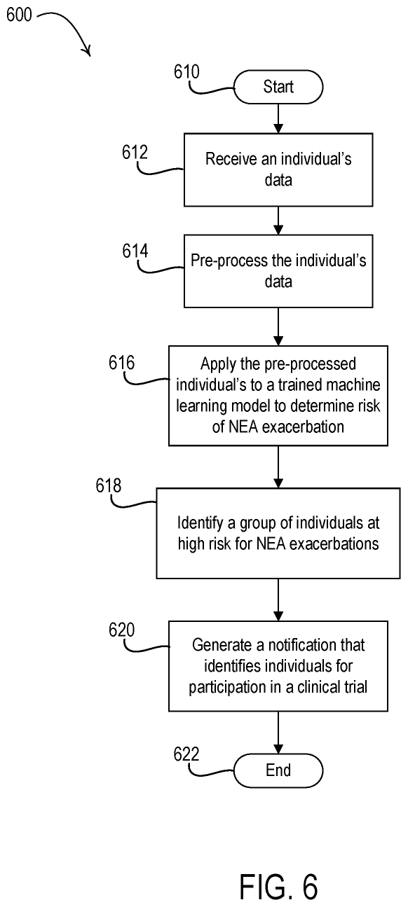

FIG. 6 illustrates an example procedure 600 performed by a computing device, such as the computing device 110 of the system 100 of FIG. 1 and/or the device 300 (e.g., as a computing device) of FIG. 3. A processor of the computing device may perform the procedure 600 to identify a group of patients for a clinical study. For example, the processor of the computing device may perform the procedure 600 to identify a group of patients (e.g., patients with NEA) that are at high risk for an asthma exacerbation and, for example, a good fit for a clinical study group. The processor may perform the procedure 600 generate a notification that identifies the patients for the clinical study. The processor may be configured to perform the procedure 600 periodically, for example, in response to an alarm or a schedule that may be configurable. Additionally or alternatively, the processor may be configured to perform the procedure 600 on command (e.g., in response to a command or prompt to perform the procedure 600) and/or in response to receiving new patient data. The procedure 600 may begin at 610.

At 612, the processor may receive patient data for one or more patients (e.g., a plurality of news patients, i.e., patients whose patient data was not used to train the model). For example, the processor may receive the patient data from one or more data sources (e.g., such as the data sources 120a-120n). The patient data may include diagnostic data relating to the patient, laboratory data relating to the patient, pharmaceutical data relating to the patient, and/or procedure data relating to the patient (e.g., as described with reference to FIG. 1). The processor may filter the data so as to only include in the data (e.g., the training data) patients with a qualifying eosinophilic blood test (i.e., test results with eosinophilic levels below a predetermined threshold). In one embodiment, only patients with a blood test indicating less than 250 eosinophils/mcL of blood are considered. Alternatively, such filtering of the data may comprise a separate step prior to 412, where the data is filtered prior to being received by the processor. The patient data may span (e.g., have been collected over) a certain period of time, for example, a period of one year prior to the qualifying eosinophilic blood test.

The format of the patient data (e.g., amount of data, the unit measures, and/or the type of data within a single group) may be different as compared to the other patient data for this patient (e.g., the new patient) and/or different from the patient data that was used to train the machine learning model. Further, as noted above, the patient data may be formatted according to one or more different codes (e.g., including different coding formats within a single patient data category, such as diagnostic data). The patient data received from the one or more data sources may be referred to as raw patient data (e.g., patient data that has yet to be preprocessed by the processor for input into the machine learning model).

At 614, the processor may pre-process the patent data for the one or more patients. For instance, the processor may pre-process the patient data (e.g., the raw patient data), for example, to homogenize the format or structure of the patient data within each category and/or to homogenize the format or structure of the patient data with the patient data that was used to train the machine learning model.

The processor may pre-process the diagnostic data. For example, the processor may pre-process the diagnostic data to determine the plurality of diagnostic categories. The diagnostic categories may include any combination of the following: an indication of whether the patient had unspecified asthma (uncomplicated) (e.g., during a baseline time period), number of asthma diagnoses, an indication of whether the patient had moderate persistent asthma with (e.g., acute) exacerbation (e.g., during a baseline time period), an indication of whether the patient had severe persistent asthma with status asthmaticus (e.g., during a baseline time period), an indication of whether the patient had mild persistent asthma (e.g., during a baseline time period), an indication of whether the patient had vasomotor and allergic rhinitis (e.g., during a baseline time period), an indication of whether the patient had a period of gestation (e.g., during a baseline time period), an indication of whether the patient had an asthma exacerbation during a baseline time period (e.g., during the previous year), etc. The indication may be binary (e.g., 1 for yes and 0 for no). In some examples, the baseline period may be one year, such as the year preceding a qualifying eosinophilic blood test.

The processor may pre-process the laboratory data. The laboratory data for a patient may include any combination of the following: average potassium level (e.g., yearly), average calcium level, average glucose level, minimum glucose level, maximum platelets level, and/or the like. In some examples, the processor may pre-process the laboratory data, for instance, using a statistical approach. In an example, one or more common tests may be applied. For example, the computing device 110 may pre-process the laboratory data and identify a subset (e.g., only a subset) of test results, such as a subset of 100 test results. In an example, lab results with no data or 0 as the result (e.g., except the tests for which 0 may be a possible result) may be removed. For example, a blood hemoglobin lab result having either no data or with 0 as the result may be removed.

The processor may pre-process pharmacy data. In an example, the processor may pre-process the pharmacy data to define any combination of the following features: count of total prescriptions; count of unique medication prescriptions (e.g., labeler and product code); an indication for each one of a plurality of medication prescriptions (e.g., 10 medication prescriptions); a count prescription of asthma rescue medication (e.g., beta2 agonist); and/or a count prescriptions of asthma anti-body medicine. In some examples, the pharmacy data be coded a National Drug Code (NDC), such as, for example, a unique 11-digit, 3-segment number, and a universal product identifier for human drugs.

The processor may pre-process the procedure data. The procedure data for a patient may include a combination of the following: an indication of whether the patient had one or more surgeries, a count of pulmonary procedures received (e.g., which may include pulmonary diagnostic testing, therapies, and/or ventilator management), a number of office visits, a number of and/or type of blood tests, an indication of whether the patient has had physical therapy, an indication and/or number of instances where the patient was admitted to the emergency department (e.g., level 4), and/or the like.

At 616, the processor may apply the pre-processed data of the one or more patients to a trained machine learning model to identify a group of patients for a clinical study, which for example, may be a subset of the plurality of patients for which patient data was received at 612. In some examples, the machine learning model may have been trained prior to 616 (e.g., in accordance with the procedure 400 of FIG. 4).

In some examples, the model may generate an indication that indicates a risk of the asthma exacerbation of the patient (e.g., the new patient, such as a new patient with NEA), for example, over a future time period (e.g., within the next month(s), year, etc.). In some examples, the indication may be binary (e.g., 1 if the patient is at risk of an asthma exacerbation, and 0 if the patient is not at risk of an asthma exacerbation). In other examples, the indication may indication whether the patient is within one of a a plurality of risk groups. The plurality of risk groups may include any combination of a low-risk group, a medium-risk group, and/or a high-risk group. The low-risk group may be defined as patients (for example, patients with NEA) having a likelihood of an asthma exacerbation below a lower threshold. The medium risk group may be defined as patients (for example, patients with NEA) having a likelihood of an asthma exacerbation above the lower threshold and below an upper threshold. The high-risk group may be defined as patients (for example, patients with NEA) having a likelihood of an asthma exacerbation above the upper threshold. In some examples, the processor may determine, using the trained model, a risk value for the patient, and compare the risk value to the lower threshold and upper threshold to determine the risk group of the patient.

At 618, the processor may identify a group of patients (for example, patients with NEA) who may be at high risk for an asthma exacerbation. As noted above, the group of patients identified by the processor at 618 may be a subset of the plurality of patients for which patient data was received by the processor at 612. For example, the processor may identify a subset of the plurality of patients that are at high risk for an asthma exacerbation and, for example, in turn a good fit for a clinical study group (e.g., for a new pharmaceutical used to treat asthma). In some examples, this group of patients may be those who are in the high-risk group.

At 620, the computing device 110 may generate a notification, for example, identifying patients for participation in a clinical trial (e.g., the group of patients, such as those patients with NEA) who may be at high risk for an asthma exacerbation). The notification may identify the subset of the plurality of patients (e.g., those patients with NEA that are at high risk for an asthma exacerbation, based on the model). The notification may provide an indication for some or all of the plurality of patients, where the indication may indicate whether the patient is a good fit for a clinical study group (e.g., for a new pharmaceutical used to treat asthma). The notification may be provided via a graphical patient interface (GUI) that is displayed on a display device (e.g., a display device of a client device, such as a client devices 130a, 130b, and/or 130c shown in FIG. 1). In some examples, the notification may be displayed for a practitioner and/or health care professional, for example, to allow them to analyze the patient's data, alert the patient, and/or adjust the treatment regimen of the patient (e.g., increase the patient's dosage, switch the patient's dosage, etc.). In other examples, the notification may be displayed for the patient so they may receive their indication of their risk of an asthma exacerbation. The procedure 600 may exit at 622.

In addition to what has been described herein, the methods and systems may also be implemented in a computer program(s), software, or firmware incorporated in one or more computer-readable media for execution by a computer(s) or processor(s), for example. Examples of computer-readable media include electronic signals (transmitted over wired or wireless connections) and tangible/non-transitory computer-readable storage media. Examples of tangible/non-transitory computer-readable storage media include, but are not limited to, a read only memory (ROM), a random-access memory (RAM), removable disks, and optical media such as CD-ROM disks, and digital versatile disks (DVDs).

While this disclosure has been described in terms of certain embodiments and generally associated methods, alterations and permutations of the embodiments and methods will be apparent to those skilled in the art. Accordingly, the above description of example embodiments does not constrain this disclosure. Other changes, substitutions, and alterations are also possible without departing from the spirit and scope of this disclosure.

What is claimed is:

1. A method for determining risk of an asthma exacerbation in a patient with non-eosinophilic asthma (NEA), the method comprising:
receiving patient data for the patient having a test result indicating an eosinophilic blood count below a predetermined threshold, wherein the patient data comprises diagnostic data relating to the patient, laboratory data relating to the patient, pharmaceutical data relating to the patient, and procedure data relating to the patient;
applying a trained model to the patient data to determine risk of an asthma exacerbation in the patient; and
generating a notification, via a display device, indicating the risk of the asthma exacerbation in the patient.

2. The method of claim 1, wherein the notification indicates that the patient is at risk of the asthma exacerbation within a time period, wherein the time period comprises one of a year or a month.

3. The method of claim 1, wherein the predetermined threshold is 250 eosinophils/mcL.

4. The method of claim 1, further comprising determining a risk group of the patient.

5. The method of claim 4, wherein the risk group is one of:
a low-risk group that is defined as being comprised of patients with NEA having the risk of the asthma exacerbation below a lower threshold,
a medium-risk group that is defined as being comprised of patients with NEA having the risk of the asthma exacerbation above the lower threshold and below an upper threshold, and
a high-risk group that is defined as being comprised of patients with NEA having the risk of the asthma exacerbation above the upper threshold.

6. The method of claim 1, further comprising pre-processing the diagnostic data to generate, for each of a plurality of diagnostic categories, an indication of whether or not the patient has been diagnosed with one or more of the plurality of diagnostic categories.

7. The method of claim 6, wherein the plurality of diagnostic categories are defined by a International Classification of Diseases 10th Revision (ICD-10) code system.

8. The method of claim 1, further comprising pre-processing the laboratory data to determine a statistical measure for one or more of a plurality of different laboratory tests for the patient.

9. The method of claim 8, wherein the statistical measure of each of the plurality of different laboratory tests for the patient comprises one or more of a minimum value for each of the plurality of different laboratory tests for the patient, a maximum value for each of the plurality of different laboratory tests for the patient, a mean for each of the plurality of different laboratory tests for the patient, and a difference between a first result and a last result for each of the plurality of different laboratory tests for the patient.

10. The method of claim 8, wherein the laboratory data is defined by a code system comprising Logical Observation Identifiers, Names, and Codes (LOINC); and
wherein pre-processing the laboratory data comprises:
removing each of the plurality of different laboratory tests for the patient with no data;
splitting the LOINC of each of the plurality of different laboratory tests for the patient into a tuple;
calculating for each LOINC, a mean of the VALUE-mean and a standard deviation; and
excluding each of the tuple with a standard deviation count greater than a predetermined number.

11. The method of claim 1, further comprising pre-processing the pharmaceutical data to determine a count of unique medication prescriptions prescribed to the patient.

12. The method of claim 11, wherein pre-processing the pharmaceutical data further comprises determining a count of total prescriptions, a count of prescriptions of asthma rescue medication, and a count of prescriptions of asthma anti-body medicine.

13. The method of claim 11, wherein pre-processing the pharmaceutical data further comprises generating, for each of a plurality of medication prescriptions, an indication of whether the patient is prescribed each of the plurality of medication prescriptions.

14. The method of claim 1, wherein the pharmaceutical data is associated with a National Drug Code (NDC).

15. The method of claim 1, further comprising pre-processing the procedure data to determine a count of procedures for the patient in an identified subset of procedure group types.

16. The method of claim 1, wherein the procedure data is associated with a Current Procedural Terminology (CPT) code.

17. The method of claim 1, further comprising pre-processing the patient data as a feature vector comprising a concatenation of the patient data and a patient metadata, wherein the patient metadata comprises demographic data of the patient.

18. The method of claim 17, wherein the demographic data comprises at least one of age, ethnicity, or gender.

19. The method of claim 1, wherein one or more of a mobile device of the patient or a healthcare provider of the patient comprise the display.

20. The method of claim 1, further comprising training the model, wherein training the model comprises:
receiving patient data for each patient of a plurality of patients each having a test result indicating an eosinophilic blood count below a predetermined threshold, wherein the patient data for each patient comprises diagnostic data relating to the patient, laboratory data relating to the patient, pharmaceutical data relating to the patient, and procedure data relating to the patient, and wherein the patient data comprises an indication of whether the patient had an NEA exacerbation during a baseline time period; and
inputting the patient data into a machine learning model to develop the trained model.

21. The method of claim 20, wherein the machine learning model comprises an Extreme Gradient Boosting (XGBoost) algorithm.

22.-55. (canceled)
